(19) United States
(12) Patent Application Publication
(10) Pub. No.: US 2009/0132009 A1
(43) Pub. Date: May 21, 2009

(54) DETERMINATION OF STIMULATION OUTPUT CAPABILITIES THROUGHOUT POWER SOURCE VOLTAGE RANGE

(75) Inventor: Nathan A. Torgerson, Andover, MN (US)

Correspondence Address: SHUMAKER & SIEFFERT , P.A, 1625 RADIO DRIVE , SUITE 300, WOODBURY, MN, 55125, US

(73) Assignee: Medtronic, Inc., Minneapolis, MN (US)

(21) Appl. No.: 11/943,858

(22) Filed: Nov. 21, 2007

(51) Int. Cl. A61N 1/00

(52) U.S. Cl. 607/61

(57) ABSTRACT

Techniques for determining whether a medical device will be able to deliver stimulation according to a particular program throughout a voltage range of a power source of the medical device are described. According to some examples, the medical device simulates a power source voltage level lower than a present voltage level of the power source, and delivers stimulation according to the program while simulating the lower power source voltage level. Whether medical device will be able to deliver stimulation according to the program when the power source is actually at the lower voltage level is determined based on an electrical parameter measured during the delivery of stimulation while simulating the lower voltage level. The simulation and determination for a program may be performed, as an example, when the program is created or modified.

### TECHNICAL FIELD

The invention relates to medical devices and, more particularly, to medical devices that include a power source and deliver electrical stimulation.

### BACKGROUND

Medical devices may be used to treat a variety of medical conditions. Some medical devices are surgically implanted within the patient, while others are connected externally to the patient receiving treatment. Some medical devices receive electrical power from batteries, such as non-rechargeable primary cell batteries or rechargeable batteries, or another power source inside the medical device, such as a supercapacitor. An electrical stimulator is an example of a medical device receives power from an internal source for delivery of a therapy to a patient.

Electrical stimulators may be used to deliver electrical stimulation therapy to patients to treat a variety of symptoms or conditions such as chronic pain, tremor, Parkinson's disease, epilepsy, urinary or fecal incontinence, sexual dysfunction, obesity, or gastroparesis. An electrical stimulator may deliver stimulation therapy via leads that include electrodes located, as examples, proximate to the spinal cord, pelvic nerves, or stomach, on or within the brain, or within the pelvic floor. In general, the electrical stimulator delivers stimulation therapy in the form of electrical pulses or substantially continuous-time signals. The electrical stimulator may be external or implanted, for example, in a chest cavity, lower back, lower abdomen, or buttocks of a patient.

A clinician selects values for a number of programmable therapy parameters in order to define the stimulation therapy to be delivered to a patient. For example, the clinician may select an amplitude, which may be a current or voltage amplitude. When therapy is delivered in the form of electrical pulses, the clinician may also select a pulse width for a stimulation waveform to be delivered to the patient as well as a rate at which the pulses are to be delivered to the patient. The clinician may also select particular electrodes within an electrode set to be used to deliver the pulses or continuous-time signal, and the polarities of the selected electrodes. The selected electrodes and their polarities may be referred to as an electrode combination or configuration. A group of parameter values may be referred to as a program in the sense that they drive the electrical stimulation therapy to be delivered to the patient.

### SUMMARY

In general, the invention is directed toward determining, for a given program, whether a medical device will be able to provide a stimulation output specified by the program throughout a voltage range of a power source of the medical device, e.g., throughout the life of a primary cell battery or between recharge cycles of a rechargeable battery or supercapacitor. When the level of charge of a power source of a medical device depletes, the ability of the medical device to deliver adequate stimulation may be impacted. For example, in embodiments that use a voltage or current regulator for delivery of stimulation, decreased power source voltage may result in an out-of-regulation condition for a given program.

By simulating a power source voltage level lower than the present power source voltage level, such as a power source voltage level a rechargeable power source may have just prior to or otherwise near full depletion, a determination as to whether the medical device will be able to deliver stimulation according to a particular program at the lower power source voltage level may be made. According to some embodiments, the medical device simulates a power source voltage level lower than a present voltage level of the power source, and delivers stimulation according to the program while simulating the lower power source voltage level. Whether medical device will be able to deliver stimulation according to the program when the power source is actually at the lower voltage level may be determined based on an electrical parameter measured during the delivery of stimulation while simulating the lower voltage level. In some embodiments for example, the output voltage of a regulator may be measured to determine whether the medical device will be able to deliver stimulation according to the program when the power source is actually at the lower voltage level.

The determination may allow a user to alter one or more therapy parameters of the program to ensure that it will be properly delivered over a range of power source voltages. In some embodiments of the invention, a user is alerted when it is determined that the medical device will not be able to deliver stimulation according to the program when the power source is at the lower voltage level. These techniques for determining whether a medical device will be able to provide a stimulation output specified by the program throughout a voltage range of a power source of the medical device may be performed, as an example, when a program is created or modified.

In one embodiment, the invention provides a method comprising simulating, in a medical device, a power source voltage level lower than a present voltage level of a power source of the medical device, and delivering electrical stimulation from the medical device according to a program while simulating the lower power source voltage level. The method further comprises determining a value of an electrical parameter within the medical device while delivering the electrical stimulation according to the program, and determining whether the medical device will be able to deliver stimulation according to the program at the lower power source voltage level based on the determined value of the electrical parameter.

In another embodiment, the invention provides a medical device comprising a power source at a current power source voltage level, a signal generator that generates electrical stimulation, and a processor. The processor configures the signal generator to simulate a power source voltage level lower than the present power source voltage level, controls the signal generator to deliver stimulation according to a program while simulating the lower power source voltage level, determines a value of an electrical parameter within the medical device during the delivery of electrical stimulation according to the program, and determines whether the medical device will be able to deliver stimulation according to the program at the lower power source voltage level based on the determined value of the electrical parameter.

In another embodiment, the invention provides a system comprising an external programming device, and an implantable medical device that comprises a power source at a current power source voltage level. The implantable medical device simulates a power source voltage level lower than the present power source voltage level, delivers stimulation according to a program while simulating the lower power source voltage level, determines a value of an electrical parameter within the medical device during the delivery of electrical stimulation according to the program, determines whether the implantable medical device will be able to deliver stimulation according to the program at the lower power source voltage level based on the determined value of the electrical parameter, and transmits an indication of the determination of whether the implantable medical device will be able to deliver stimulation according to the program at the lower power source voltage level to the external programming device.

In another embodiment, the invention provides a medical device comprising means for simulating a power source voltage level lower than a present voltage level of a power source of the medical device, means for delivering electrical stimulation according to a program while simulating the lower power source voltage level, means for determining a value of an electrical parameter within the medical device while delivering the electrical stimulation according to the program, and means for determining whether the medical device will be able to deliver stimulation according to the program at the lower power source voltage level based on the determined value of the electrical parameter.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic perspective view of an example therapy system, which includes an electrical stimulator coupled to a stimulation lead.

FIG. 2 is a block diagram illustrating various components of an example electrical stimulator.

FIG. 3 is a block diagram illustrating various components of an example stimulation interface.

FIG. 4 is a block diagram illustrating various components of another example of an electrical stimulator including a testing module.

FIG. 5 is a block diagram illustrating various components of yet another example of an electrical stimulator including a test capacitor module.

FIG. 6 is a flow chart illustrating an example method of determining whether a medical device will be able to deliver constant current stimulation according to a program at a lower battery voltage level.

FIG. 7 is a flow chart illustrating an example method of determining whether a medical device will be able to deliver constant voltage stimulation according to a program at a lower battery voltage level.

FIG. 8 is a flow chart illustrating an example method of determining whether a medical device will be able to deliver stimulation according to a program at a lower battery voltage level based on capacitor droop.

### DETAILED DESCRIPTION

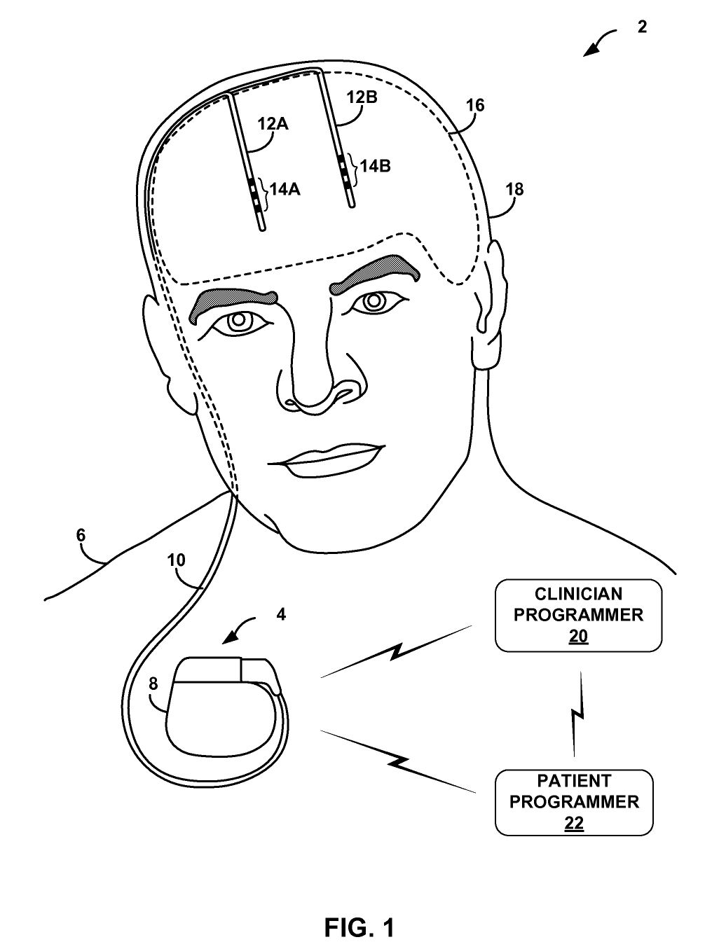

FIG. 1 is a schematic perspective view of therapy system 2, which includes medical device 4. Medical device 4 may be either implantable or external. In the example of FIG. 1, medical device 4 has been implanted in patient 6. For example, medical device 4 may be subcutaneously implanted in the body of patient 6 (e.g., in a chest cavity, lower back, lower abdomen, buttocks, or cranium of patient 6). Patient 6 will ordinary be a human patient. In some cases, however, the invention may be applied to a non-human patient.

In the embodiment illustrated in FIG. 1, medical device 4 is an electrical stimulator and provides a programmable stimulation signal (e.g., in the form of electrical pulses or substantially continuous-time signals) that is delivered to patient 6 by implantable medical lead 10 and, more particularly, via one or more stimulation electrodes carried by lead 10. Medical device 4 may also be referred to as a pulse or signal generator. In the example of FIG. 1, the distal end of lead 10 is bifurcated and includes two segments 12A and 12B. Segments 12A and 12B each include an electrode array 14A and 14B, respectively. At least some of the electrodes of arrays 14A and 14B may be stimulation electrodes to deliver a stimulation signal from medical device 4 to patient 6. In some embodiments, lead 10 may also carry one or more sense electrodes to permit electrical medical device 4 to sense electrical signals from patient 6. In various embodiments, medical device 4 may be coupled to one or more leads, which may or may not be bifurcated.

A proximal end of lead 10 may be both electrically and mechanically coupled to medical device 4 either directly or indirectly (e.g., via a lead extension). In particular, conductors disposed in the lead body may electrically connect stimulation electrodes adjacent to the distal end of lead 10 (e.g., the electrodes of electrode arrays 14A and 14B) to medical device 4. Lead 10 may also include one or more lead anchors, e.g., tines, adhesives, sutures, or any other suitable anchors (not shown in FIG. 1), along its lead body to help prevent migration of lead 10.

In the example shown in FIG. 1, lead 10 extends to brain 16 of patient 6, e.g., through cranium 18 of patient 6. Medical device 4 may deliver deep brain stimulation (DBS) or cortical stimulation (CS) therapy to patient 6 via the electrodes of arrays 14A and 14B of lead 10 to treat any of a variety of movement disorders, including tremor, Parkinson's disease, spasticity, epilepsy, or dystonia. However, the invention is not limited to the configuration of lead 10 and electrodes arrays 14A and 14B shown in FIG. 1, or to the delivery of DBS or CS therapy.

Therapy system 2 may be useful in other stimulation applications, including pelvic floor stimulation, spinal cord stimulation, cortical surface stimulation, neuronal ganglion stimulation, gastric stimulation, peripheral nerve stimulation, or subcutaneous stimulation. Such therapy applications may be targeted to a variety of disorders such as chronic pain, peripheral vascular disease, angina, headache, tremor, depression, Parkinson's disease, epilepsy, urinary or fecal incontinence, sexual dysfunction, obesity, or gastroparesis. Further, therapy system 2 may be useful in non-neurostimulation contexts. For example, medical device 4 may be used to deliver stimulation to a target muscle tissue site via leads to, for example, provide functional electrical stimulation or cardiac stimulation, e.g., cardiac pacing. In various embodiments, therapy system 2 may deliver therapy to any nerve or other tissue site in patient 6.

Therapy system 2 also may include a clinician programmer 20 and a patient programmer 22. Clinician programmer 20 may be a handheld computing device that permits a clinician to program stimulation therapy for patient 6 via a user interface, e.g., using input keys and a display. For example, using clinician programmer 20, the clinician may specify stimulation parameters, i.e., create programs, for use in delivery of stimulation therapy. Clinician programmer 20 may support telemetry (e.g., radio frequency telemetry) with medical device 4 to download programs and, optionally, upload operational or physiological data stored by medical device 4. In this manner, the clinician may periodically interrogate medical device 4 to evaluate efficacy and, if necessary, modify the programs or create new programs. In some embodiments, clinician programmer 20 transmits programs to patient programmer 22 in addition to or instead of medical device 4.

Like clinician programmer 20, patient programmer 22 may be a handheld computing device. Patient programmer 22 may also include a display and input keys to allow patient 6 to interact with patient programmer 22 and medical device 4. In this manner, patient programmer 22 provides patient 6 with a user interface for control of the stimulation therapy delivered by medical device 4. For example, patient 6 may use patient programmer 22 to start, stop or adjust electrical stimulation therapy. In particular, patient programmer 22 may permit patient 6 to adjust stimulation parameters of a program such as duration, current or voltage amplitude, pulse width and pulse rate. Patient 6 may also select a program, e.g., from among a plurality of stored programs, as the current program to control delivery of stimulation by medical device 4.

In some embodiments, medical device 4 delivers stimulation according to a group of programs at any given time. Each program of such a program group may include respective values for each of a plurality of therapy parameters, such as respective values for each of amplitude (e.g., current or voltage amplitude), pulse width, pulse rate and electrode combination. Medical device 4 may interleave pulses or other signals according to the different programs of a program group, e.g., cycle through the programs, to, for example, simultaneously treat different symptoms or provide a combined therapeutic effect. In such embodiments, clinician programmer 20 may be used to create programs, and assemble the programs into program groups. Patient programmer 22 may be used to adjust stimulation parameters of one or more programs of a program group, and select a program group, e.g., from among a plurality of stored program groups, as the current program group to control delivery of stimulation by medical device 4.

Medical device 4, clinician programmer 20, and patient programmer 22 may communicate via cables or a wireless communication, as shown in FIG. 1. Clinician programmer 20 and patient programmer 22 may, for example, communicate via wireless communication with medical device 4 using RF telemetry techniques known in the art. Clinician programmer 20 and patient programmer 22 also may communicate with each other using any of a variety of local wireless communication techniques, such as RF communication according to the 802.11 or Bluetooth specification sets, infrared communication, e.g., according to the IrDA standard, or other standard or proprietary telemetry protocols. Each of clinician programmer 20 and patient programmer 22 may include a transceiver to permit bi-directional communication with medical device 4.

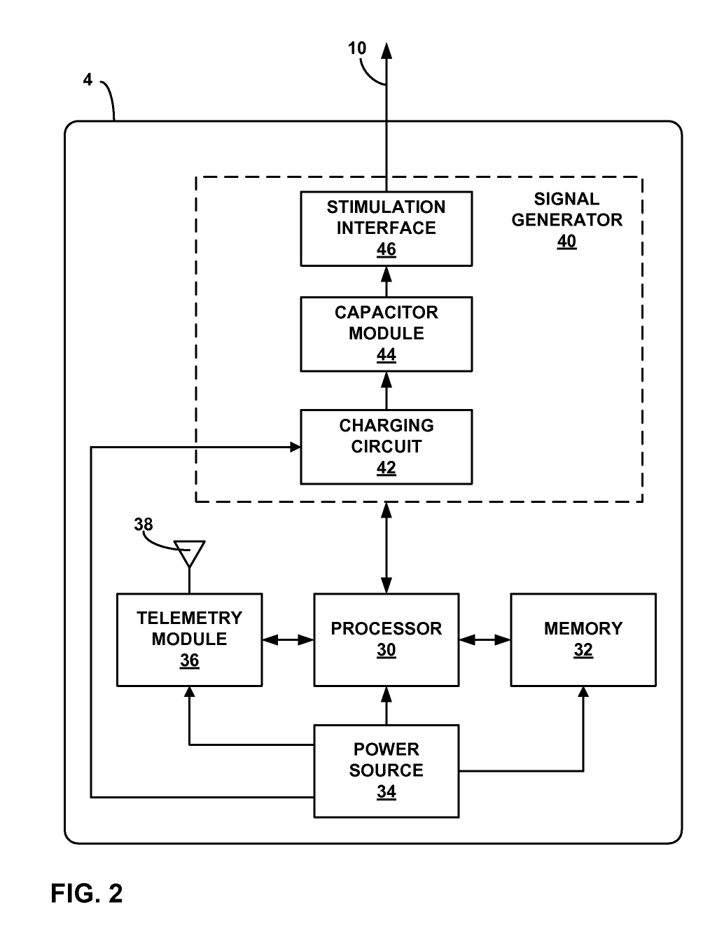

FIG. 2 is a block diagram illustrating various components of medical device 4. In the example of FIG. 2, medical device 4 includes processor 30, memory 32, power source 34, telemetry module 36, antenna 38, and signal generator 40. Telemetry module 36 may permit communication with clinician programmer 20 and patient programmer 22 to, for example, receive new programs or program groups, or adjustments to programs or program groups.

Processor 30 may include one or more microprocessors, digital signal processors (DSPs), application-specific integrated circuits (ASICs), field-programmable gate arrays (FPGAs), or other digital logic circuitry. Processor 30 controls operation of medical device 4, e.g., controls signal generator 40 to deliver stimulation therapy according to a selected program or group. For example, processor 30 may control signal generator 40 to deliver electrical signals with current or voltage amplitudes, pulse widths (if applicable), and rates specified by one or more stimulation programs. Processor 30 may also control signal generator 40 to deliver the stimulation signals via subsets of the electrodes of arrays 14A and 14B with polarities, the subsets and polarities specified as electrode combinations or configurations by one or more programs.

At any given time, processor 30 may control signal generator 40 to deliver stimulation according to a selected one or more of a plurality of programs or program groups stored in memory 32. Memory 32 may include any magnetic, electronic, or optical media, such as random access memory (RAM), read-only memory (ROM), electronically-erasable programmable ROM (EEPROM), flash memory, or the like. Memory 32 may store program instructions that, when executed by processor 30, cause the processor to perform the functions ascribed to it and medical device 4 herein.

Telemetry module 36 may include a transceiver to permit bi-directional communication between medical device 4 and each of clinician programmer 20 and patient programmer 22. Telemetry module 36 may include an antenna 38 that may take on a variety of forms. For example, antenna 38 may be formed by a conductive coil or wire embedded in a housing associated with medical device 4. Alternatively, antenna 38 may be mounted on a circuit board carrying other components of electrical stimulator 4 or take the form of a circuit trace on the circuit board.

Power source 34 may be a non-rechargeable primary cell battery or a rechargeable battery and may be coupled to power circuitry. However, the invention is not limited to embodiments in which the power source is a battery. In another embodiment, as an example, power source 34 may comprise a supercapacitor. In some embodiments, power source 34 may be rechargeable via induction or ultrasonic energy transmission, and include an appropriate circuit for recovering transcutaneously received energy. For example, power source 34 may be coupled to a secondary coil and a rectifier circuit for inductive energy transfer.

Signal generator 40 produces an electrical stimulation signal in accordance with a program based on control signals from processor 30. As shown in FIG. 2, signal generator 40 may include a charging circuit 42, a capacitor module 44, and a stimulation interface 46. Charging circuit 42 selectively, e.g., based on signals from processor 30, applies energy from power source 34 to capacitor module 44 to charge the capacitor module for delivery of a stimulation signal, e.g., pulse. For delivery of pulses, charging circuit 42 may control the pulse rate based on signals from the processor by controlling the rate at which capacitor module 44 is recharged. In addition to capacitors, capacitor module 44 may include switches. In this manner, capacitor module 44 may be configurable, e.g., based on signals from processor 30, to store a desired voltage for delivery of a stimulation at a voltage or current amplitude specified by a program. For delivery of stimulation pulses, switches within capacitor module 44 may control the width of the pulses based on signals from processor 30.

Stimulation interface 46 conditions charge from capacitor module 44 to produce an electrical stimulation signal, e.g., a pulse, under control of processor 30 for application to at least some electrodes of electrode arrays 14A and 14B carried by lead 10. Stimulation interface 46 may control the voltage or current amplitude of the signal based on signals from processor 30. Stimulation interface 46 may also control to which electrodes of arrays 14A and 14B the stimulation signal is provided, and the polarities of the electrodes, based on signals from processor 30.

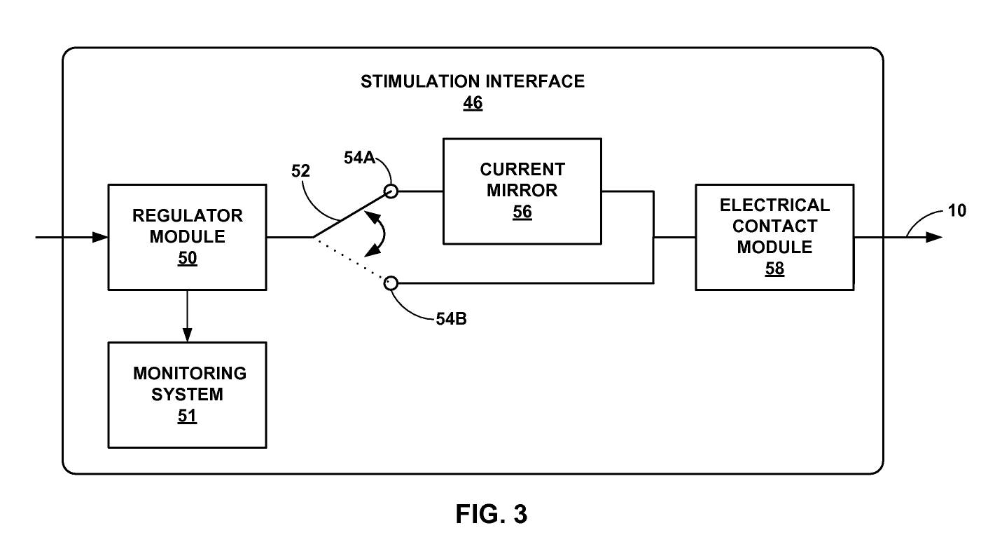

FIG. 3 is a block diagram illustrating various components of stimulation interface 46 according to one example embodiment. In the example illustrated in FIG. 3, stimulation interface 46 includes regulator module 50, monitoring system 51, current mirror 56, and electrical contact module 58. Regulator module 50 may be, for example, a voltage regulator that outputs a substantially constant voltage at a programmable value. The input voltage to regulator module 50 may be higher than the output voltage value to provide adequate “headroom” for regulator module 50 to maintain the desired output voltage value.

In the illustrated embodiment, stimulation interface 46 is selectively, e.g., based on a signal from processor 30, able to deliver either constant voltage or constant current stimulation pulses to patient 6. However, the invention is not limited to embodiments in which both constant voltage and constant current pulses are available. Other embodiments may provide only constant voltage pulses, or only constant current pulses. Furthermore, as indicated above, the invention is not limited to embodiments in which stimulation is in the form of pulses.

In the example embodiment illustrated by FIG. 3, when therapy is delivered to patient 6 using constant voltage mode, processor 30 may actuate switch 52 to connect the output of regulator module 50 to node 54B, which bypasses current mirror 56. In this manner, the output of regulator module 50, a constant voltage at the amplitude specified by a stimulation program, is output to lead 10 via electrical contact module 58.

When stimulation is delivered to patient 6 using constant current mode, regulator module 50 is coupled to current mirror 56 via switch 52 as controlled by processor 30. When constant current stimulation is delivered to patient 6, processor 30 may actuate switch 52 to connect the output of regulator module 50 to node 54A of current mirror 56. In this manner, the output of regulator module 50 is provided as the input voltage for current mirror 56. Current mirror 56 will output a constant current, to lead 10 via electrical contact module 58, at an amplitude specified by a stimulation program, based on the input voltage from regulator module 50.

Electrical contact module 58 may include a plurality of switches that may be controlled by processor 30. Each of the switches within electrical contact module 48 may be coupled to a conductor within lead 10 to allow processor 30 to control therapy delivery to a selected subset of electrodes according to an electrode configuration specified by a current stimulation program. However, the invention is not limited to embodiment that include an electrical contact module comprising a plurality of switches to selectively multiplex the output of a regulator module and/or current mirror across a plurality of electrodes. In other embodiments, for example, each electrode of a lead 10 may be associated with a respective voltage or current source, e.g., regulator and/or current mirror. Accordingly, in some embodiments, selection of electrodes and polarities by processor 30 according to an electrode configuration specified in a stimulation program may involve selection of a voltage or current source by the processor, instead of or in addition to switching the source across selected electrodes.

Regulator module 50 receives an input signal from capacitor module 44 (FIG. 2). Capacitor module 44 may include a plurality of capacitors and a switching array. The capacitors of capacitor module 44 may be configured into various configurations, including various charge and discharge configurations, using the switching array under control of processor 30. In this manner, processor 30 may control the charge and discharge configurations of the capacitors to produce a desired output of capacitor module 44, which is input into regulator module 50.

As one example, if a pair of capacitors is charged (e.g., configured in a charge configuration) across power source 34 in parallel and subsequently discharged (e.g., configured in a different discharge configuration) across a load in series, the output voltage of the capacitor pair will be double that of power source 34. In contrast, if a capacitor pair is charged across battery 34 in series and subsequently discharged across a load in parallel, the output voltage of the capacitor pair will be one half the voltage of power source 34. However, the example of capacitor pairs is used solely for purposes of illustration and is not intended to limit the invention. According to the invention, capacitor module 44 may include one or more capacitor pairs, capacitor triplets, capacitor octets, any other types of capacitor configurations, or any other number of capacitors.

In some embodiments, a maximum stack capacitor arrangement may be used to test if medical device 4 will be able to deliver stimulation according to a particular stimulation program given the present voltage level of power source 34. Maximum stack refers to a combination of charge and discharge configurations that result in the greatest possible multiple of the present power source voltage. The maximum stack arrangement allowed may vary based on power source type (e.g., non-rechargeable primary cell versus rechargeable) as well as stimulation mode (e.g., constant current versus constant voltage). When a maximum stack arrangement is used, the output of capacitor module 44 is as large as possible for the present power source voltage level, power source type, and stimulation mode.

Monitoring system 51 may detect whether medical device 4 is able to deliver stimulation according to a present program. In the example illustrated in FIG. 3, monitoring system 51 is coupled to regulator module 50, and detects whether medical device 4 is able to deliver stimulation according to the present program by detecting out of regulation conditions. In other embodiments, monitoring system 51 may be coupled to one or more of processor 30, regulator module 50, current mirror 56, and electrical contact module 58. System monitor 51 may measure a value of an electrical parameter within signal generator 40, such as a voltage, to detect whether medical device 4 is able to deliver stimulation according to the present program

As one example, system monitor 51 may measure the voltage input into regulator module 50, and processor may compare the measured voltage to a threshold voltage. The threshold voltage may represent a minimum input voltage necessary to produce a stimulation signal according to the present program. As another example, system monitor 51 may measure a voltage output of regulator module 50, and processor 30 may compare the measured voltage to the desired stimulation amplitude specified by the present program. In this manner, system monitor 51 and processor 30 may detect if regulator module 50 is unable to produce an output signal that will support the presently selected stimulation program.

Monitoring system 51 may additionally or alternatively measure a voltage drop across regulator module 50, and processor 30 may detect whether there is sufficient headroom based on the measured voltage drop. Headroom refers to the voltage difference between the input of regulator module 50 and the output of regulator module 50. If the headroom is insufficient, e.g., the voltage drop is below a threshold value, regulator module 50 may not be able to provide a stimulation signal with a constant amplitude at the value specified by the present program. For example, the amplitude of a stimulation pulse may droop over the duration of the pulse.

If processor 30 determines that medical device 4 is, or will be, unable to deliver stimulation according to selected program based on a parameter measured by monitoring system 51, processor 30 may report the determination over a telemetry channel via telemetry module 36. For example, if processor 30 detects an out of regulation condition based on an electrical parameter value measured by monitoring system 51, processor 30 may report the out of regulation condition via telemetry module 36. In response to such a report, a user may wish to modify the stimulation program, e.g., decrease an amplitude, of the stimulation signal. In embodiments in which power source 34 is rechargeable, a user may wish to recharge power source 34 in response to such a report. The report may be provided to the user, e.g., clinician or patient, via one of programmers 20, 22, or another external device. In addition to an indication that medical device is, or will be, unable to deliver therapy according to the program, the external device may also provide recommendations to the user about how to respond, e.g., decrease an intensity of stimulation, recharge the power source, or the like.

In some embodiments, when stimulation is initiated according to a new program, or an intensity of the stimulation signal is increased, e.g., the programmed amplitude is increased, processor 30 configures capacitor module 44 to the maximum stack arrangement for delivery of the stimulation. Delivering stimulation using a maximum stack arrangement at these times may allow monitoring system 51 and processor 30 to detect if the medical device 4 will be able to effectively deliver stimulation conforming the specified stimulation parameter values of the new, or newly modified program at the present power source voltage level. If the medical device is able to effectively deliver the specified stimulation when the maximum stack arrangement is used, processor 30 may reconfigure the capacitors of capacitor module 50 to the most efficient stack arrangement for the program. The most efficient stack arrangement for a given program may allow the input of regulator module 50 to be greater than, but as close as possible to, the sum of the desired output of regulator module 50 and the required headroom. Adjusting capacitor module 50 in this manner may prolong the life of power source 34. Due to the time required to identify the most efficient stack arrangement, it may not be necessary or practical to adjust capacitor module 50 if the program is transient. Instead, capacitor module 50 may be ramped down after no programming changes have been received by processor 30, e.g., via telemetry module 36, for a threshold period of time.

Processor 30 may also configure capacitor module 50 to simulate a power source voltage level lower than the current voltage level of the power source. This may allow processor 30 and system monitor 51 to determine, e.g., predict, if medical device 4 will continue to be able to deliver the stimulation specified by the particular program as power source 34 depletes. Testing the program, i.e., delivering stimulation according to the program, while simulating the lower power source voltage level may be performed after the test at maximum stack. As an example, processor 30 may cause medical device 4, e.g., configure signal generator 40, to simulate the lower power source voltage level by configuring capacitor module 50 to multiply a voltage of power source 34 by an amount less than the battery voltage is multiplied by using the maximum stack arrangement.

As one example, the lower power source voltage level may be simulated using a capacitor stack arrangement that stores a voltage that is multiples one half of the power source voltage less than the voltage stored by the maximum stack arrangement. In one embodiment, for example, the maximum stack arrangement of capacitor module 50 may store a voltage that is four times that of power source 34. When simulating the lower battery voltage, processor 30 may configure capacitor module 50 to store a voltage that is three and one half times that of power source 34. These example multiplier values for capacitor module 50 may be useful, for example, in embodiments in which power source 34 is a rechargeable battery and medical device 4 is delivering constant voltage stimulation.

As another example, the maximum stack arrangement of capacitor module 50 may store a voltage that is five times that of power source 34. When simulating the lower power source voltage, processor 30 may configure capacitor module 50 to store a voltage that is four and one half times that of power source 34. These example multiplier values for capacitor module 50 may be useful, for example, in embodiments in which power source 34 is a non-rechargeable, primary cell battery, and medical device 4 is delivering constant voltage stimulation.

As yet another example, the maximum stack arrangement of capacitor module 50 may store a voltage that is two and one half times that of power source 34. When simulating the lower power source voltage, processor 30 may configure capacitor module 50 to store a voltage that is two times that of power source 34. These example multiplier values for capacitor module 50 may be useful, for example, in embodiments in which power source 34 is rechargeable battery and medical device 4 is delivering constant current stimulation.

The one half of a power source voltage decrement interval for lower power source voltage simulation and maximum stack multiplication factors are discussed above solely for purposes of providing examples. In other embodiments, the maximum stack arrangement may multiply the voltage of power source 34 by any appropriate factor, and the stack arrangement used in the lower power source voltage level simulation may multiply the voltage of power source 34 by any amount lower than the maximum stack multiplication factor. For example, in some embodiments the power source voltage decrement interval may be less than one half of a power source voltage and, in some embodiments, less than one fourth of the power source voltage.

A small power source voltage decrement interval, e.g., less than one half of a power source voltage and, in some embodiments, less than one fourth of a power source voltage, may be used in embodiments in which the power source comprises a non-rechargeable, primary cell battery. The voltage of a primary cell battery remains relatively constant throughout most of the life of the battery, and then gradually decreases toward the end of the life of the battery. In contrast, the voltage of a rechargeable battery may fluctuate daily and the magnitudes of the fluctuations may be larger. A small power source voltage decrement interval may be useful in giving an advance warning that a selected set of therapy parameters will cause an out of regulation condition if the battery voltage of a primary cell battery decreases by a small amount over a relatively long period of time, e.g., months or years.

By using a capacitor arrangement that multiplies the voltage of power source 34 by an amount less than the maximum stack configuration multiplies the voltage of power source 34, a lower voltage is applied to regulator 50. This lower voltage may simulate a voltage delivered that will be delivered to regulator module 50 when a maximum or efficient stack arrangement is used, e.g., during normal operation, in the future when power source 34 has a lower power source voltage, i.e., when the level of charge of power source 34 has decreased. In this manner, processor 30 and system monitor 51 may detect whether the medical device will be able to correctly deliver stimulation according to a selected stimulation program, in the future when the power source 34 voltage level is lower, and the lower voltage is therefore applied to regulator module 50.

Processor 30 may determine whether medical device will be able to correctly deliver stimulation according to a program at a simulated lower power source voltage level in the manner described previously with respect to determine whether medical device will be able to correctly deliver stimulation according to a program at a present battery charge level, i.e., based on an electrical parameter value within signal generator 40 or elsewhere within medical device 4 measured by system monitor 51. For example, system monitor 51 may measure a voltage input into regulator 50, a voltage output of regulator 50, and/or a voltage drop across regulator 50. Processor 30 may determine whether medical device will be able to correctly deliver stimulation according to a program at a simulated lower power source voltage level by determining whether delivery of stimulation according to the program while simulating the lower power source voltage level causes an out of regulation condition, e.g., determining whether regulator 50 is able to provide the required amplitude.

Processor 30 may report whether medical device 4 will be able to correctly deliver the stimulation specified by a program at the simulated lower battery charge level to a user via telemetry module 36, e.g., by transmitting an indication or information to one or both of programming devices 20, 22, in the manner discussed above. As discussed above, programming device may also provide recommendations to the user about how to avoid ineffective stimulation as power source 34 depletes, e.g., by suggesting a decrease in the intensity of stimulation, recharging power source 34 before its charge level reaches the simulated, lower power source voltage level, replacing power source 34 or medical device 4 when its voltage depletes to a certain value, or the like.

In some embodiments of the invention, the simulation of the lower power source voltage level may be performed during a programming session by a clinician, e.g., using clinician programmer 20. The simulation may be performed during testing of new programs or modified programs during such a programming session. Based on the results of such simulations, the clinician may choose programs that medical device 4 will be able to support throughout the useable life of power source 34. If power source 34 is rechargeable, power source 34 may be fully charged during the programming session. If power source 34 is not fully charged during the programming session, the simulated lower power source voltage level may be too conservative, e.g., lower than a power source voltage level that power source 34 will reach between charging events.

The delivery of ineffective stimulation may be prevented by determining whether a medical device will continue to be able to deliver stimulation as specified by a stimulation program as power source 34 depletes. This may be particularly important for patients receiving stimulation for movement disorders. Some of these patients may become physically disabled if the stimulation intensity is less than is necessary for effective reduction of movement disorder symptoms, making it difficult to correct the situation. If stimulation stops working properly for a patient receiving pain therapy, the patient may be able to alert the clinician, recharge the power source, etc. In contrast, if stimulation stops working properly for a movement disorder patient, the patient may be unable to do so. Additionally, a patient receiving pain therapy may feel tingling sensations during stimulation delivery and notice when the stimulation is interrupted. A patient receiving DBS to treat a movement disorder may not notice that stimulation has been interrupted until symptoms occur, at which point the patient may be unable to correct the situation.

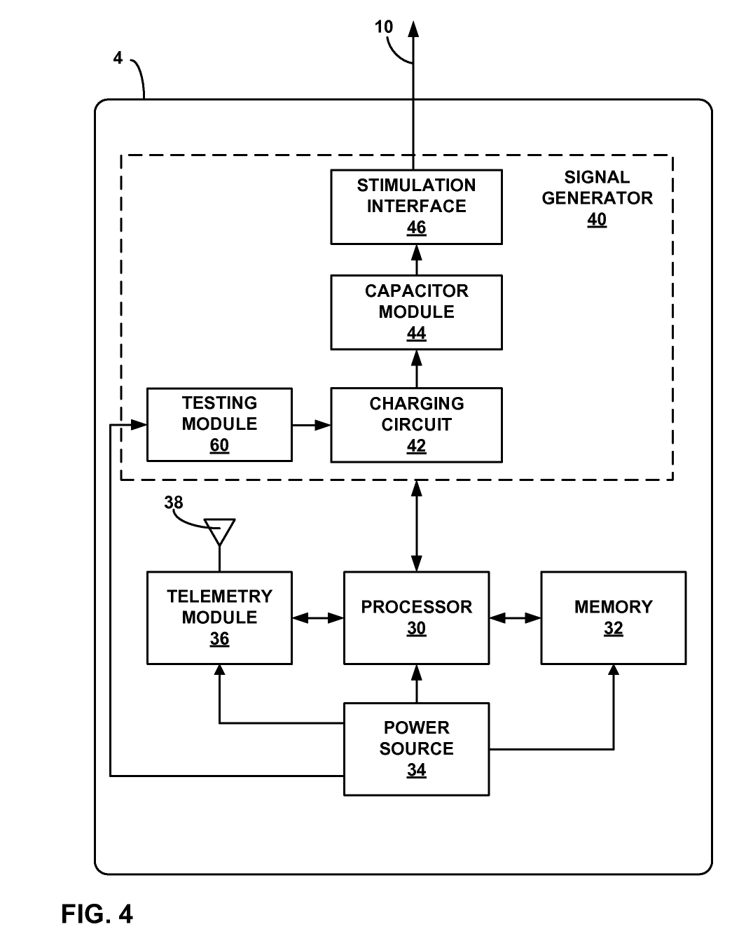

FIG. 4 is a block diagram illustrating various components of another embodiment of medical device 4 including a testing module 60 coupled to charging circuit 42. Testing module 60 may operate under the control of processor 30 to lower the voltage inputted into charging circuit 42, which will also lower the voltage inputted into regulator module 50. For example, processor 30 may control testing module 60 to simulate a battery charge level lower than a current level of charge of battery 34.

In some embodiments, testing module 60 may include, for example, a voltage divider or an impedance control module. In embodiments including a voltage divider, processor 30 may activate the voltage divider, e.g., via a switching mechanism of testing module 60, to simulate a lower power source voltage level. The voltage divider may input a fraction of the voltage from power source 34 into charging circuit 42 and, consequentially, regulator 50. In embodiments in which testing module 60 includes an impedance control module, processor 30 may control the impedance control module, e.g., via a switching mechanism of testing module 60, to simulate a lower power source voltage level. The impedance control module may increase the impedance between battery 34 and charging circuit 34, which decreases the voltage input into charging circuit 34 and, consequentially, regulator 50.

Testing module 60 may be used to simulate the lower battery charge level as an alternative or in addition to capacitor module 44. In the embodiment illustrated in FIG. 4, testing module 60 is positioned between battery 34 and charging circuit 42. In other embodiments, testing module 60 may be positioned between charging circuit 42 and capacitor module 44, or between capacitor module 44 and regulator module 50.

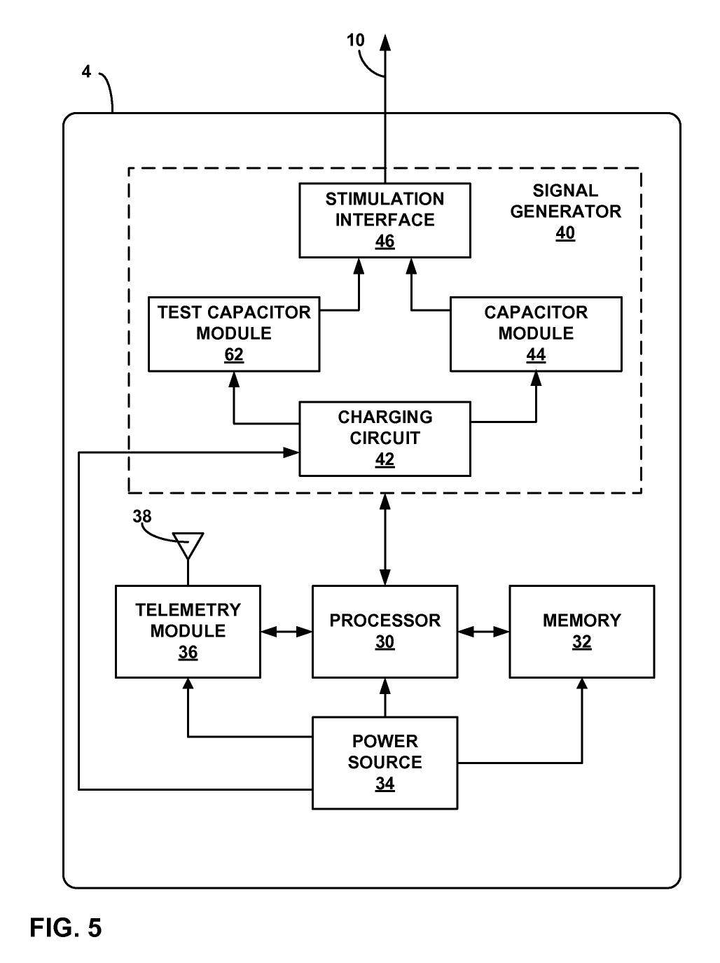

FIG. 5 is a block diagram illustrating various components of another embodiment of medical device 4 including test capacitor module 62 coupled to charging circuit 42 and stimulation interface 46. Like capacitor module 44, test capacitor module 62 may include a plurality of capacitors. The capacitors of test capacitor module 62 may have smaller capacitance (i.e., hold less charge) than the capacitors of capacitor module 44. For example, in some embodiments, the capacitance of the capacitors of capacitor module 44 may be approximately 10 microfarads (μF) to approximately 68 μF. In some embodiments, the capacitors of test capacitor module 62 may be approximately 1 microfarads (μF) to approximately 10 μF.

Processor 30 may select test capacitor module 62 for charging from power source 34 to simulate a lower power source voltage level. Because test module 62 has less capacitance, it may have a more pronounced, e.g., faster, droop when loaded during discharge than capacitor module 44. The droop of test capacitor module 62 during delivery of stimulation according to a particular program may indicate whether regulator module 50 will be out of regulation during delivery of stimulation according to the program when battery has a lower voltage level in the future.

To measure the droop of test capacitor module 62, a voltage level of the capacitors of test capacitor module 62 may be measured at a given time after discharge, e.g., by a system monitor 51. Processor 30 may compare the measured voltage to a threshold value. If the measured voltage is below the threshold, i.e., if the droop was greater than a threshold, processor 30 may determine that the medical device will not be able to deliver stimulation according to the program at the simulated lower power source voltage level. As described previously, processor 30 may send an indication of this determination to a programming device (e.g., clinician programmer 20 and/or patient programmer 22) via telemetry module 36.

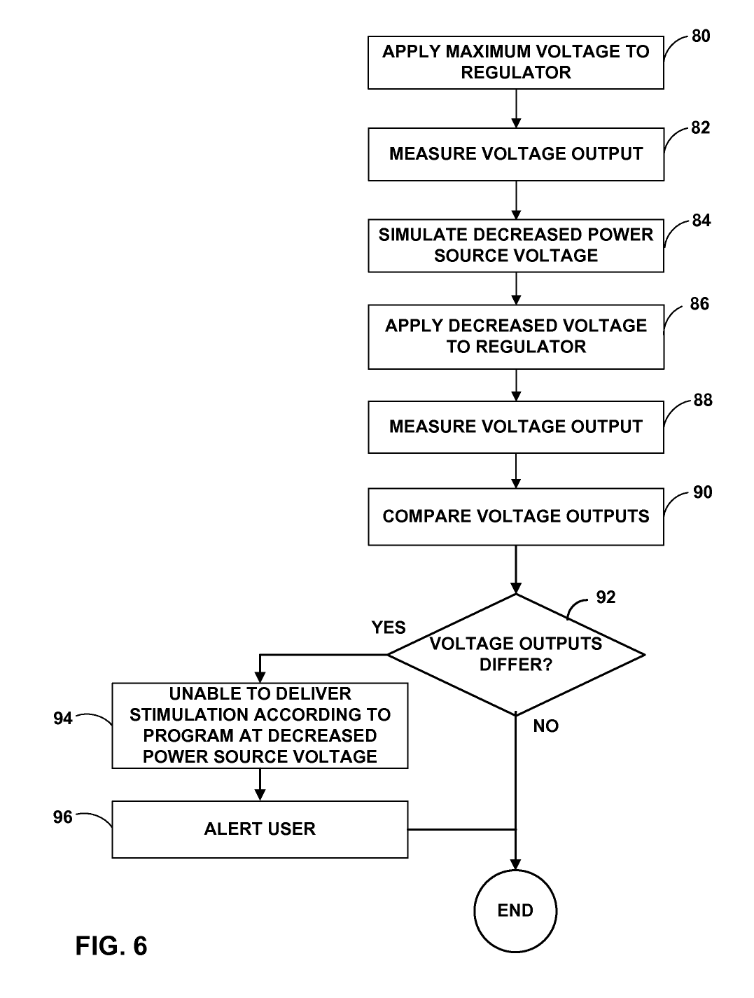

FIG. 6 is a flow diagram illustrating an example method of determining whether a medical device will, in the future, be able to deliver constant current stimulation according to a program at a lower power source voltage level. A maximum voltage is applied to regulator 50 using a maximum stack arrangement of the capacitors of capacitor module 44 (80) during a first delivery of stimulation according to the program. An output voltage of regulator 50 is measured by system monitor 51 (82). Next, a battery voltage lower than the present battery voltage is simulated (84) during a second delivery of stimulation according to the program. As described previously, the simulation may be performed, for example, using a less than maximum stack capacitor arrangement, voltage divider, and/or impedance control module. During the simulation, i.e., during the second delivery of stimulation according to the program, a decreased voltage is applied to regulator 50 (86). System monitor 51 measures an output of regulator 50 while the decreased voltage is inputted into regulator 50 (88).

Processor 30 may compare the voltage output of regulator 50 when the maximum stack arrangement was used to the voltage output of regulator 50 when the decreased power source voltage was simulated (90). If the two outputs differ, e.g., by a threshold amount (92), processor 30 determines that medical device 4 will be unable to deliver stimulation according to the program when the power source is at the lower voltage level, e.g., by determining that an out of regulation condition is detected at the simulated decreased power source voltage level (94). Processor 30 may alert a user of this determination via telemetry module 36 (96).

In other embodiments, system monitor 51 may measure an output current of current mirror 56 for comparison to a desired current value provided by processor 30. Processor 30 may determine whether medical device 4 will be able to deliver stimulation according to the program when the power source is at the lower voltage level based on the comparison. In such embodiments, it may be not necessary to measure an output of regulator 50 at the maximum stack arrangement.

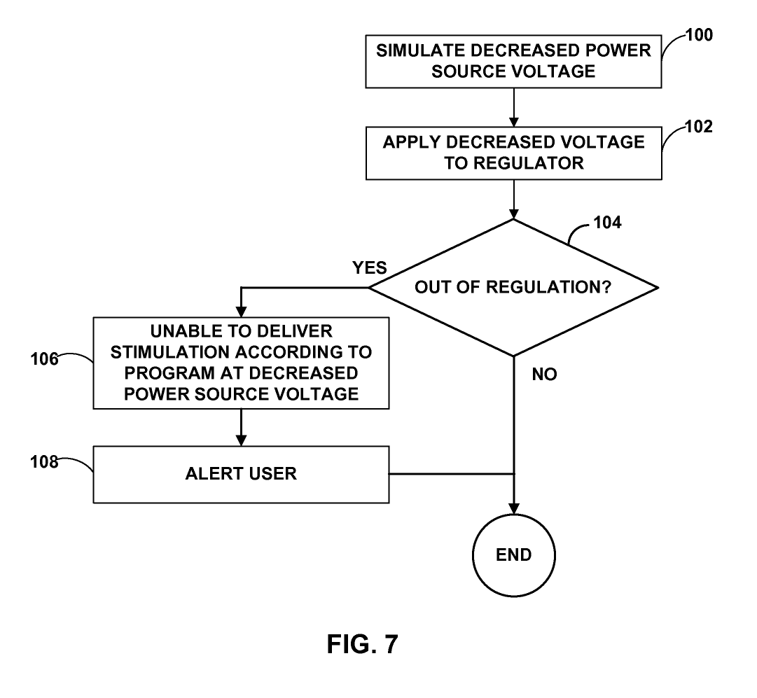

FIG. 7 is a flow diagram illustrating an example method of determining whether a medical device will, in the future, be able to deliver constant voltage stimulation according to a program at a lower power source voltage level. A power source voltage lower than the present battery voltage is simulated during delivery of stimulation according to the program (100). As described previously, the simulation may be performed, for example, using a less than maximum stack capacitor arrangement, voltage divider, and/or impedance control module. During the simulation, a decreased voltage is applied to regulator 50 (102). System monitor 51 may detect whether regulator 50 is having problems outputting a desired voltage, i.e., is out of regulation during delivery of stimulation according to the program (104). For example, system monitor 51 may measure an input of, output of, and/or voltage drop across regulator 50 to detect whether regulator 50 is having problems outputting a desired voltage. If system monitor 51 does detect an out of regulation condition, processor 30 determines that medical device 4 will be unable to deliver stimulation according to the program when the power source is at the lower voltage level (106). Processor 30 may alert a user of this determination via telemetry module 36 (108).

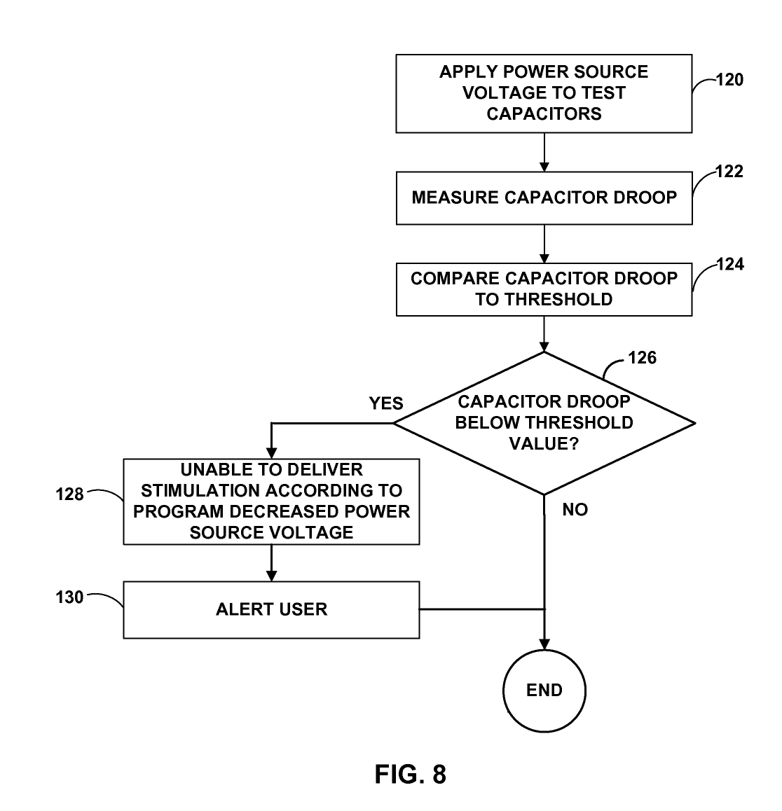

FIG. 8 is a flow chart illustrating an example method of determining whether a medical device will, in the future, be able to deliver constant voltage stimulation according to a program at a lower power source voltage level by measuring capacitor droop. Processor 30 selects test capacitor module 62, and a voltage from power source 34 is applied to test capacitor module 62 to simulate a battery voltage lower than the present battery voltage (120). Capacitor droop may be measured by measuring the charge of the capacitors of test capacitor module 62 at a time after discharge (122). Processor 30 may compare the measurement to a threshold value (124). If the measured value is below the threshold value (126), processor 30 may determine that medical device 4 will be unable to deliver stimulation according to the program when the power source is at the lower voltage level (128). Processor 30 may alert a user of this determination via telemetry module 36 (130).

Various embodiments of the invention have been described. However, the invention is not limited to the described embodiments. For example, although described with reference to embodiments in which a voltage or current source for delivery of stimulation includes a voltage regulator, the invention is not so limited. Other embodiments may additionally or alternatively include a current regulator, or no regulator. These and other embodiments are within the scope of the following claims.

1. A method comprising:
simulating, in a medical device, a power source voltage level lower than a present voltage level of a power source of the medical device;
delivering electrical stimulation from the medical device according to a program while simulating the lower power source voltage level;
determining a value of an electrical parameter within the medical device while delivering the electrical stimulation according to the program; and
determining whether the medical device will be able to deliver stimulation according to the program at the lower power source voltage level based on the determined value of the electrical parameter.

2. The method of claim 1, wherein simulating a power source voltage level lower than a present voltage level of a power source of the medical device comprises applying a reduced input voltage to a regulator module of the medical device for the delivery of the electrical stimulation according to the program.

3. The method of claim 2, wherein applying the reduced voltage to the regulator module of the medical device comprises introducing a voltage divider between the power source and the regulator module.

4. The method of claim 2, wherein applying the reduced voltage to the regulator module of the medical device comprises introducing an impedance between the power source and the regulator module.

5. The method of claim 1, wherein delivering the electrical stimulation comprises charging a capacitor module from the power source, and simulating the lower power source voltage level comprises configuring the capacitor module to simulate the lower power source voltage level.

6. The method of claim 1,
wherein simulating the lower power source voltage level comprises selecting a second capacitor module from among a first capacitor module and the second capacitor module, wherein the second capacitor module has a lower capacitance than the first capacitor module,
wherein delivering electrical stimulation while simulating the lower power source voltage level comprises charging the second capacitor module from the power source,
wherein determining a value of an electrical parameter within the medical device while delivering the electrical stimulation comprises measuring droop of the second capacitor module while delivering the electrical stimulation, and
wherein determining whether the medical device will be able to deliver stimulation according to the program at the lower power source voltage level comprises:
comparing the droop measurement to a threshold value; and
determining whether the medical device will be able to deliver stimulation according to the program at the lower power source voltage level based on the comparison.

7. The method of claim 1, wherein determining a value of an electrical parameter within the medical device comprises measuring an output voltage of a regulator module of the medical device.

8. The method of claim 1, further comprising sending an indication to a user of whether the medical device is able to deliver electrical stimulation according to the program at the lower power source voltage level based on the determination.

9. The method of claim 8, wherein sending the indication comprises sending the indication to a programming device, wherein the programming device presents the indication to the user.

10. The method of claim 1, wherein simulating the low power source voltage level comprises simulating the low power source voltage level in response to receipt of the program.

11. The method of claim 1, wherein simulating the low power source voltage level comprises simulating the low power source voltage level in response to modification of the program.

12. A medical device comprising:
a power source at a present power source voltage level;
a signal generator that generates electrical stimulation; and
a processor that configures the signal generator to simulate a power source voltage level lower than the present power source voltage level, controls the signal generator to deliver stimulation according to a program while simulating the lower power source voltage level, determines a value of an electrical parameter within the medical device during the delivery of electrical stimulation according to the program, and determines whether the medical device will be able to deliver stimulation according to the program at the lower power source voltage level based on the determined value of the electrical parameter.

13. The medical device of claim 12, wherein the signal generator comprises a regulator module, and the processor configures the signal generator to apply a reduced input voltage to the regulator module to simulate the lower power source voltage level.

14. The medical device of claim 13, wherein processor configures the signal generator to include a voltage divider between the power source and the regulator module to simulate the lower power source voltage level.

15. The medical device of claim 13, wherein processor configures the signal generator to include an impedance between the power source and the regulator module to simulate the lower power source voltage level.

16. The medical device of claim 12, wherein the signal generator comprises a capacitor module that is charged from the power source for delivery of electrical stimulation, wherein the processor configures the capacitor module to simulate the lower power source voltage level.

17. The medical device of claim 12,
wherein the signal generator comprises a first capacitor module and a second capacitor module, the second capacitor module having a lower capacitance than the first capacitor module, and
wherein the processor selects the second capacitor module for charging from the power source for delivery of the electrical stimulation, receives a measurement of droop of the second capacitor module during the delivery of the electrical stimulation, compares the droop measurement to a threshold value, and determines whether the medical device will be able to deliver stimulation according to the program at the lower power source voltage level based on the comparison.

18. The medical device of claim 12, wherein the processor receives a measurement of an output voltage of a regulator module of the medical device, and determines whether the medical device will be able to deliver stimulation according to the program at the lower power source voltage level based on the measurement.

19. The medical device of claim 12, further comprising a transceiver that communicates with a programming device, wherein the processor sends an indication via the transceiver of whether the medical device will be able to deliver stimulation according to the program at the lower power source voltage level based on the determined value of the electrical parameter.

20. The medical device of claim 12, wherein the medical device comprises an implantable medical device.

21. The medical device of claim 12, wherein the power source comprises a battery.

22. The medical device of claim 12, wherein the power source is rechargeable.

23. A system comprising:
an external programming device; and
an implantable medical device comprising a power source at a present power source voltage level, wherein the implantable medical device simulates a power source voltage level lower than the present power source voltage level, delivers stimulation according to a program while simulating the lower power source voltage level, determines a value of an electrical parameter within the medical device during the delivery of electrical stimulation according to the program, determines whether the implantable medical device will be able to deliver stimulation according to the program at the lower power source voltage level based on the determined value of the electrical parameter, and transmits an indication of the determination of whether the implantable medical device will be able to deliver stimulation according to the program at the lower power source voltage level to the external programming device.

24. A medical device comprising:
means for simulating a power source voltage level lower than a present voltage level of a power source of the medical device;
means for delivering electrical stimulation according to a program while simulating the lower power source voltage level;
means for determining a value of an electrical parameter within the medical device while delivering the electrical stimulation according to the program; and
means for determining whether the medical device will be able to deliver stimulation according to the program at the lower power source voltage level based on the determined value of the electrical parameter.
